(19) United States
(12) Patent Application Publication
(10) Pub. No.: US 2005/0147526 A1
(43) Pub. Date: Jul. 7, 2005

(54) Process for washing and disinfecting hands and fingers

(76) Inventor: Iwao Hishida, Toyonaka (JP)

Correspondence Address: LORUSSO, LOUD & KELLY, 3137 Mount Vernon Avenue, Alexandria, VA, 22305, US

(21) Appl. No.: 11/010,939

(22) Filed: Dec. 14, 2004

(30) Foreign Application Priority Data
Dec. 24, 2003 (JP) 2003-426489

(51) Int. Cl. A01N 1/00

(52) U.S. Cl. 422/28; 422/292; 134/198

(57) ABSTRACT

The present invention aims at providing a process and an apparatus for washing and disinfecting hands, fingers and arms for a shortened time. That is, the present invention provides a process for washing and disinfecting hands, fingers and arms, which process comprises contacting hands, fingers and arms with a disinfectant used in a state of flowing water and then drying them to wash and disinfect the hands, fingers and arms, and also provides an apparatus for carrying out said process.

### INCORPORATION BY REFERENCE

The present application claims priority from Japanese application JP 2003-426489 filed on Dec. 24, 2003, the content of which is hereby incorporated by reference into this application.

### FIELD OF THE INVENTION

The present invention relates to a process and an apparatus for washing and disinfecting hands, fingers and arms, which are used in medical operation.

### BACKGROUND OF THE INVENTION

In disinfection of human bodies, particularly disinfection of hands, fingers and the like of those engaged in medical care such as doctors before operation, not only transient flora but also resident flora on the skin have been subject to disinfection, and furthermore continuous effects of disinfection have been requested.

For example, as disclosed in “Manual of Sterilization and Disinfection, Color Edition” page 39 publiched by Ishiyaku Publishers, Inc., Japan, supervising editor: Masakazu Tuzuki, first edition, tenth copy published on Oct. 15, 2000, the conventional disinfection of hands and fingers of those engaged in medical care such as doctors before operation has been carried out by using a soap, a scrubbing agent, and an alcohol-containing preparation, more particularly and roughly by (1) roughly washing with a soap, then (2) brushing by use of a scrubbing agent, and furthermore (3) rubbing with an alcohol-containing preparation.

Concretely, for example, there have been carried out by about once or twice repeating the steps such as roughly washing with a soap, then brushing with a scrubbing agent, and washing away from fingertips to elbows with sterile water, then wiping by pushing with a sterile towel to remove the water, and then further rubbing hands and fingers on from fingertips to arms with about 3 ml of a disinfectant.

However, in the conventional disinfection process as mentioned above, there is a defect that physical washing by a brush with a scrubbing agent of high concentration is carried out frequently and strongly and hence injures the skin and causes chap of hands in many cases. Moreover, it is considered that bacteria are firmly fixed and grow proliferously at the rough portions of hands.

Furthermore, in the above conventional disinfection, more concretely, for example, there have been carried out handlings such as (1) roughly washing with a soap, (2) brushing by use of a scrubbing agent with a sterile brush for about 3 to 4 minutes to disinfect, (3) washing away the scrubbing agent with sterile water, (4) furthermore brushing by use of a scrubbing agent with a fresh sterile brush similarly again for about 3 to 4 minutes to disinfect, (5) again washing away the scrubbing agent with sterile water, and furthermore (6) rubbing hands and fingers with a disinfectant about twice or three times at intervals of about 30 seconds. However, as mentioned above, the conventional disinfection process has had problems that complicated steps are required and moreover much time is consumed.

Furthermore, JP-A-7-165571 discloses a quick-drying disinfecting composition which is described as nonsticky and which contains ethanol, and a cationic bactericide such as benzalkonium chloride, benzethonium chloride, chlorhexidine gluconate, alkylisoquinolinium bromide or the like, and a wetting agent such as glycerine, polyethylene glycol, propylene glycol, butylene glycol or the like, and a cyclic or linear silicone or a fatty acid ester of a polyhydric alcohol.

Furthermore, JP-A-2000-86408 discloses a quick-drying bactericide composition for disinfecting hands and fingers which is described as having higher safety and continuing uniform effects and which contains as essential components 95% ethanol solution, and a gelatinous resin polymer, and glucomannan remaining in gelatinous state. However, the gelatinous resin polymer is left as solid residue, and hence it is difficult to completely dry and remove the bactericide.

Furthermore, JP-A-2002-517358 discloses a skin-disinfecting bactericidal composition which is described as being used for disinfecting surgery site in carrying out surgical operation and which contains as essential components iodine, and ethyl alcohol, and a water-soluble gel. However, the water-soluble gel is contained and hence it is difficult to completely dry and remove the disinfectant.

### SUMMARY OF THE INVENTION

The present invention resolves the challenges and problems of the prior art as mentioned above and highly simplifies washing and disinfection. The main object of the present invention is to simplify disinfection of the human bodies including hands, fingers, and arms of those engaged in medical care such as doctors in the field relating to medical care including operation by those engaged in medical care.

The present invention resolves said challenges and furthermore highly simplifies washing and disinfection. The present invention relates to a process for washing and disinfecting hands, fingers and arms in medical operation, which process comprises contacting hands, fingers and arms with a disinfectant used in a state of flowing water and then drying them to wash and disinfect the hands, fingers and arms, and also relates to an apparatus for washing and disinfecting hands, fingers and arms in medical operation by carrying out said process.

Other objects, features and advantages of the invention will become apparent from the following description of the embodiments of the invention taken in conjunction with the accompanying drawings.

### BRIEF DESCRIPTION OF THE DRAWINGS

FIG. 1 shows a schematic view of the appearance of the washing and disinfecting apparatus according to one working embodiment of the present invention.

FIG. 2 shows a schematic view of a portion of the inside of the washing and disinfecting apparatus according to one working embodiment of the present invention.

FIG. 3 shows a schematic view of the appearance of a portion of the washing and disinfecting apparatus according to one working embodiment of the present invention.

FIG. 4 shows a schematic view of the washing and disinfecting process or apparatus in use according to one working embodiment of the present invention.

FIG. 5 shows a schematic view of the washing and disinfecting process or apparatus in use according to one working embodiment of the present invention.

### DESCRIPTION OF THE NUMERALS IN THE DRAWINGS

1: washing machine

2: opening and closing door

3: washing liquid

4: plastic tank

5: exhaust hose

6: exhaust opening

7: power switch

8: power indication light

9: washing time-setting switch

10: drying time-setting switch

11: sensor switch

12: fingertip passing position

13: actuation indication light

14: spray nozzle

15: hot air drying portion

16: hot air-generating machine

17: tab

### DETAILED DESCRIPTION OF THE INVENTION

The washing and disinfecting process and washing and disinfecting apparatus of the present invention are used, when those engaged in medical care such as doctors carry out medical practice including operation.

The physical objects to be washed and disinfected by the washing and disinfecting process and washing and disinfecting apparatus of the present invention are human bodies including hands, fingers and arms of those engaged in medical care such as doctors.

In the washing and disinfecting process of the present invention, the sequence for washing and disinfecting is not particularly limited, as long as hands, fingers and arms are contacted with a disinfectant used in the state of flowing water and then dried.

In the washing and disinfecting apparatus of the present invention, the member constitution of the apparatus is not particularly limited, as long as the apparatus is used for washing and disinfecting hands, fingers and arms by contacting hands, fingers and arms with a disinfectant used in the state of flowing water and then drying them.

The washing and disinfecting apparatus of the present invention is required to be provided with, for example, a tank for storing the disinfectant, a washing machine, and a drier.

Said washing machine is furthermore required to be provided with a water-flowing device including a faucet, a spray nozzle or the like.

Also, said drier is furthermore required to be provided with a hot air-generating machine.

In the washing and disinfecting process and washing and disinfecting apparatus of the present invention, as the disinfectant to be used, a disinfectant containing water can be used as such in the state of flowing water, or a disinfectant can be used by first preparing a concentrate thereof containing no water in advance and then mixing it with water such as flowing water just before washing and disinfecting.

As a washing period of time in the washing and disinfecting process and washing and disinfecting apparatus of the present invention, a time of about 5 seconds to 1 minute is sufficient for washing and even a short time of about 5 to 30 seconds makes washing possible.

As a drying period of time in the washing and disinfecting process and washing and disinfecting apparatus of the present invention, a time of about 2 to 3 minutes is sufficient for drying and even a short time of about 5 to 10 seconds makes drying possible.

The disinfectant used in the washing and disinfecting process and washing and disinfecting apparatus of the present invention is not particularly limited, and medical agents certified as drug medicines or quasi-drugs can be used, and also publicly known disinfectants may be used. Furthermore, for example, a medical agent containing 40-80% by weight of an alcohol and 0.5-5% by weight of a moisturing agent and water can be used.

In addition, said alcohol is not particularly limited and can include ethyl alcohol, propyl alcohols such as isopropyl alcohol and the like. Isopropyl alcohol is preferable from the viewpoints of improvement in disinfecting effect and decrease in chap of hands.

The content of the alcohol in said disinfectant is, for example, 40-80% by weight and can be also 45-60% by weight. When the content is less than these lower limits, disinfecting effect tends to be low, and when the content is more than these upper limits, the skin highly tends to be chapped.

The kind of said moisturing agent is not particularly limited and can include at least one component selected from, for example, glycerine and glycols such as 1,3-butanediol. Use of the moisturing agent tends to exhibit an effect of decreasing a defatting power by an alcohol and more preventing injuring of the skin.

A content of the moisturing agent in the disinfectant composition is, for example, 0.5 to 5% by weight. When the content is less than the lower limit value, the effect of preventing injuring of the skin tends to be low, and when the content is more than the upper limit value, the composition tends to be easily sticky.

The disinfectant used in the washing and disinfecting process and washing and disinfecting apparatus of the present invention may contain an additional bactericide. The additional bactericide is not particularly limited, and can include, solely or in combination, at least one component selected from gluconic acid, chlorohexidine gluconate, benzalkonium chloride (alkylbenzyldimethylammonium chloride wherein alkyl is —C₈H₁₇ to —C₁₈H₃₇), benzethonium chloride, and trade name, Tego 51 (ampholytic surface active agent), and the like. Incorporating the additional bactericide tends to increase bactericidal effect.

The content of said additional bactericide in the disinfectant is not particularly limited and can be, for example, about 0 to 2% by weight, and also 0.5 to 1% by weight.

Said disinfectant can be also applied as a disinfected material, for example, by absorbing the disinfectant into an absorber such as synthetic fibers or natural fibers such as nonwoven fabric.

Thus, said handlings in washing and disinfecting as in the prior art can be saved to attain the object of the present invention.

### DESCRIPTION OF PREFERRED EMBODIMENTS

Hereinafter the present invention is described in detail by way of examples.

### EXAMPLE 1

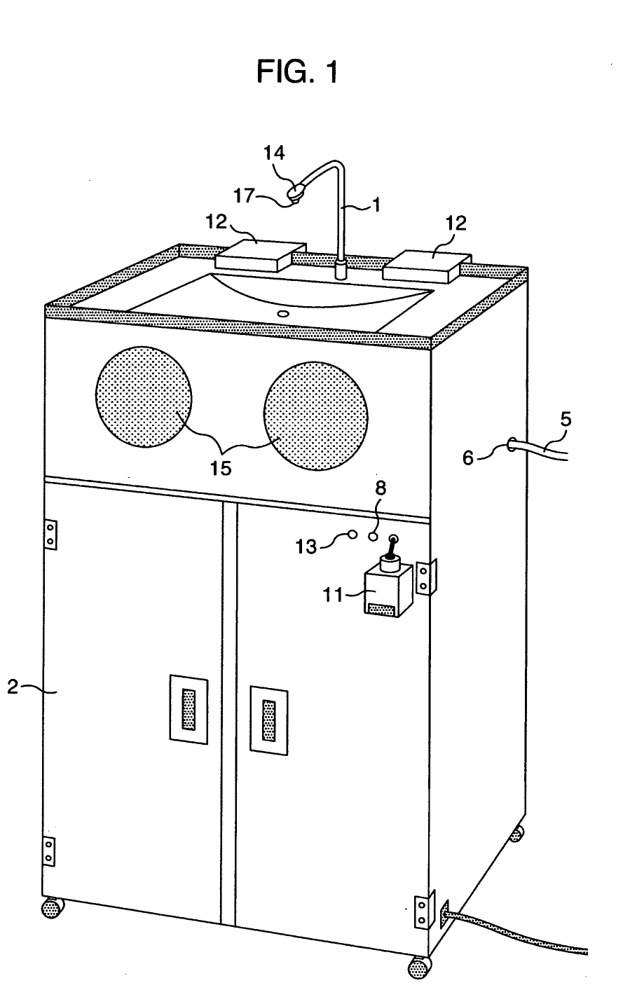

First, hands and fingers were roughly washed with sterile water (test method A) or hands and fingers were not washed with sterile water (test method B), and then from the hands and fingers to arms were washed for about 10 seconds by flowing a disinfectant consisting of the undermentioned composition out of a faucet of a washing machine. Thereafter, from the hands and fingers to the arms were inserted into a hot air drying portion of the washing machine and were dried for about 5 to 10 seconds. Refer to FIG. 1 to FIG. 5.

Composition of the disinfectant used:

| | |
|---|---|
| Chlorohexidine gluconate | 0.1% by weight |
| Isopropyl alcohol | 50% by weight |
| Glycerine | 3% by weight |
| Water | 46.9% by weight |

The step of washing with the above disinfectant and drying (disinfection step) was carried out respectively once, twice and three times, and the number of bacteria on the skin was measured. Measurement of the number of bacteria was carried out as follows.

Method for measuring the number of bacteria:

(1) Culture medium used

A culture medium for common viable bacteria examination and for hands and fingers examination (standard agar medium) was used.

(2) Subjects

Three subjects were used. Subject A was a woman of age 20-29, subject B was a woman of age 40-49, and subject C was a woman of age 30-39.

(3) Test methods

[Test Method A]

(i) The untreated flat of a hand (only one hand) was pushed directly (stamped) on the agar medium marked with the outline of hand and fingers. Then, the medium, after the stamping, was incubated for 48 hours at 36° C., and the colony count, which developed and appeared, was counted. → (“Untreated”)

(ii) Hands were washed with tap water, and then the remaining water was wiped, and similarly to (i) only the hand on the same side as in (i) was stamped, and the colony count was counted. Washing time with tap water was not particularly set. → (“After washing with water”)

(iii) The above disinfectant was flown out of the faucet until the entire hands got wet, and the hands were dried with hot air, and then stamping was carried out similarly to (i), and the colony count was counted. → (“After disinfected once”)

(iv) Again, the above disinfectant was flown out of the faucet until the entire hands got wet, and the hands were dried with hot air, and then stamping was carried out similarly to (i), and the colony count was counted. → (“After disinfected twice”)

(v) Again, the above disinfectant was flown out of the faucet until the entire hands got wet, and the hands were dried with hot air, and then stamping was carried out similarly to (i), and the colony count was counted. → (“After disinfected three times”)

[Test Method B]

(i) The untreated flat of a hand (only one hand) was pushed directly (stamped) on the agar medium marked with the outline of hand and fingers. Then, the medium, after the stamping, was incubated for 48 hours at 36° C., and the colony count, which developed and appeared, was counted. → (“Untreated”)

(ii) The above disinfectant was flown out of the faucet until the entire hands got wet, and the hands were dried with hot air, and then stamping was carried out similarly to (i), and the colony count was counted. → (“After disinfected once”)

(iii) Again, the above disinfectant was flown out of the faucet until the entire hands got wet, and the hands were dried with hot air, and then stamping was carried out similarly to (i), and the colony count was counted. → (“After disinfected twice”)

(iv) Again, the above disinfectant was flown out of the faucet until the entire hands got wet, and the hands were dried with hot air, and then stamping was carried out similarly to (i), and the colony count was counted. → (“After disinfected three times”)

The results thus obtained are shown in Table 1 and Table 2.

TABLE 1
Test method A (Untreated → Washing with water →
Disinfected once to three times)

| | | After | | | After |
|---|---|---|---|---|---|
| | | washing | After | After | disinfected |
| | | with | disinfected | disinfected | three |
| Subjects | Untreated | water | once | twice | times |
| A | 70 | 55 | 0 | 1 | 0 |
| B | Countless | 39 | 246 | 0 | 0 |
| C | Countless | 49 | 50 | 1 | 0 |

Notes:
The numerals in the Table represent colony counts. “Countless” represents a colony count of at least 300.

TABLE 2
Test method B (Untreated → No washing with water →
Disinfected once to three times)

| | | After dis- | After dis- | After dis- |
|---|---|---|---|---|
| | | infected | infected | infected |
| Subjects | Untreated | once | twice | three times |
| A | 60 | 1 | 0 | 1 |
| B | 50 | 0 | 0 | 0 |
| C | Countless | 0 | 0 | 0 |

Notes:
The numerals in the Table represent colony counts. “Countless” represents a colony count of at least 300.

### EXAMPLE 2

Other embodiments of the washing and disinfecting process and apparatus of the present invention are described below.

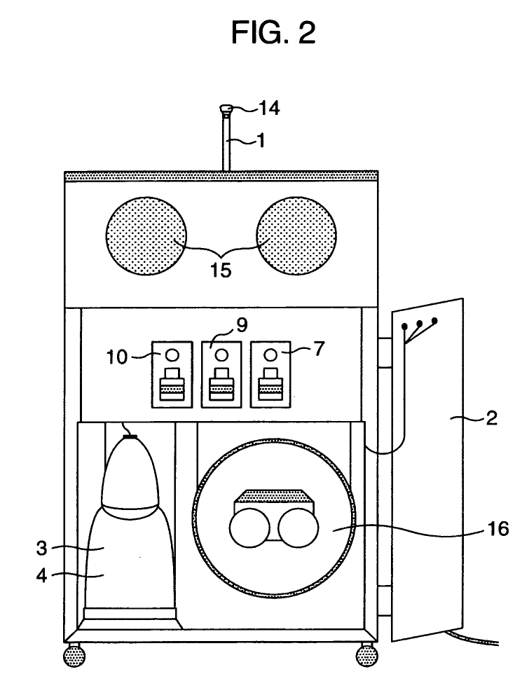

1. Opening and closing doors (2) of washing machine (1) were opened, and washing liquid (3) was poured into plastic tank (4). (Refer to FIG. 1 and FIG. 2.)

2. Exhaust hose (5) was installed into exhaust opening (6). In addition, the exhaust hose could be used on both the right side and left side.

3. When power switch (7) was turned on, an upper light of the power switch was lighted up, and also power indication light (8) on the front face of the washing machine was lighted up.

4. A washing period of time and a drying period of time were set respectively with a washing period of time-setting switch (9) and a drying period of time-setting switch (10). For example, the washing time could be set at 30 seconds and the drying time could be set at 180 seconds, and the setting times could be changed as necessary.

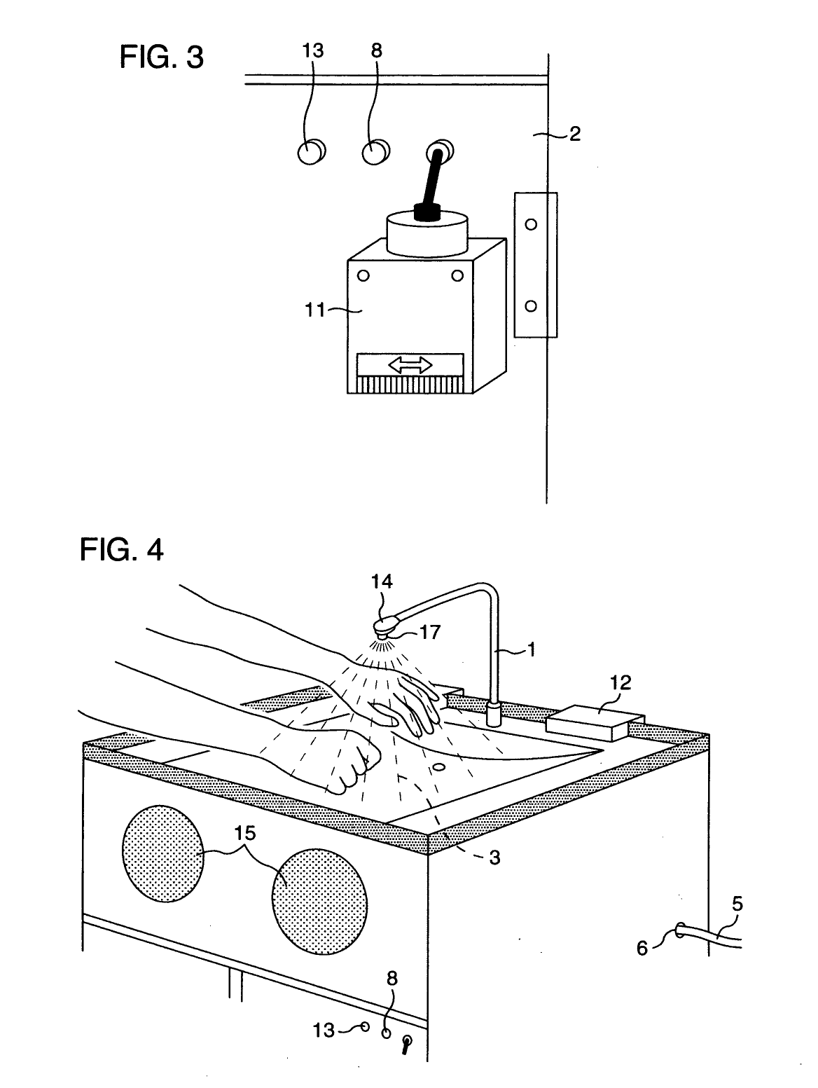

5. When the doors of the washing machine were closed and fingertips were passed through fingertip passing position (12) controlled with sensor switch (11), an actuation indication light (13) was lighted up and a washing liquid was sprayed from spray nozzle (14) at the top of the washing machine, and therefore hands were washed. In addition, when a tab (17) at a front edge of the spray nozzle was turned, the spray state was changed, and hence the spray could be used in a suitable state. Furthermore, the height of the spray nozzle could be changed. Moreover, a faucet could be used in place of the spray nozzle. Refer to FIG. 3 and FIG. 4.

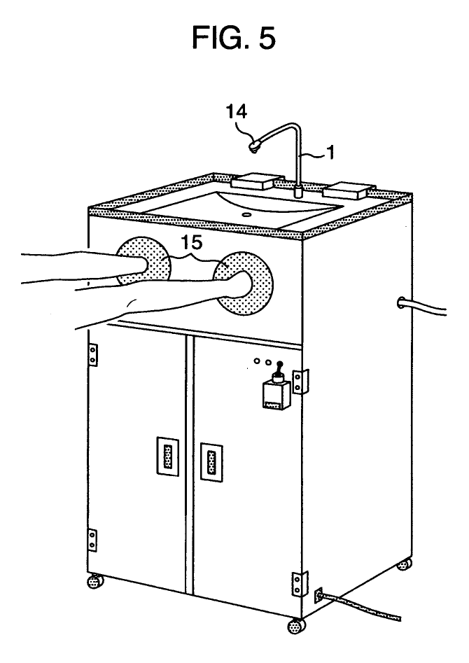

6. After the predetermined time, the spray was stopped. When hands were inserted into hot air drying portion (15), hot air was generated from hot air-generating machine (16) and was blown out from both sides of the hands for a predetermined period of time, and therefore the hands were dried. In addition, the drying can be stopped on the way by passing fingertips through the fingertip passing position (12) again. Refer to FIG. 5.

### Industrial Usability

The washing and disinfecting process and apparatus of the present invention are applicable to washing and disinfecting of human bodies including hands, fingers and arms of those engaged in medical care such as doctors in the field of medical care such as operation by those engaged in medical care.

### Advantages of the Invention

In the present invention, brushing step using a brush is not carried out, and hence the skin is not injured and little chap of hands is caused, and therefore it is seldom caused that bacteria are firmly fixed and grow proliferously at the chapped portions of hands.

Furthermore in the present invention, it is not necessary to carry out to wipe a sterile water or the like with a cloth as used in the prior art, and therefore the present invention has no needlessness and moreover can shorten washing and disinfecting time highly.

Moreover in the present invention, after washing and disinfecting, a disinfectant can be dried and removed in a short time, and hence neither solid material nor gelatinous material remains.

The present invention can disinfect transient flora and resident flora such as Staphylococcus aureus, Bacillus subtilis, environmentally extractive bacteria, MRSA, O-157, Serratia, Pseudomonas aeruginosa, and the like.

It should be further understood by those skilled in the art that although the foregoing description has been made on embodiments of the invention, the invention is not limited thereto and various changes and modifications may be made without departing from the spirit of the invention and the scope of the appended claims.

1. A process for washing and disinfecting hands, fingers and arms in medical operation, which process comprises contacting hands, fingers and arms with a disinfectant used in a state of flowing water and then drying them to wash and disinfect the hands, fingers and arms.

2. An apparatus for washing and disinfecting hands, fingers and arms in medical operation by contacting hands, fingers and arms with a disinfectant used in a state of flowing water and then drying them to wash and disinfect the hands, fingers and arms.

3. The apparatus as set forth in claim 2, which is provided with:
a tank for storing the disinfectant;
a washing machine; and
a drier.

4. The apparatus as set forth in claim 3, wherein said washing machine is provided with at least one water-flowing device selected from the group consisting of a faucet and a spray nozzle.
